(19) United States
(12) Patent Application Publication
(10) Pub. No.: US 2017/0196607 A1
(43) Pub. Date: Jul. 13, 2017

(54) IMPLANT FOR FIXING FEMUR

(71) Applicant: GSM KOREA CO., LTD., Gwangju (KR)

(72) Inventors: Jung-Won SHIN, Gwangju (KR); Gyeong-Su KIM, Seoul (KR); Ji-Yeong JANG, Gwangju (KR); Seo-Yeon PARK, Gyeonggi-do (KR); Na-Young KIM, Jeollanam-do (KR)

(21) Appl. No.: 15/341,859

(22) Filed: Nov. 2, 2016

(30) Foreign Application Priority Data
Jan. 7, 2016 (KR) 10-2016-0002201

(51) Int. Cl. A61B 17/82; A61B 17/80

(52) U.S. Cl. CPC A61B 17/82; A61B 17/80; A61B 17/8061; A61B 2017/681

(57) ABSTRACT

Provided is an implant for fixing a femur, the implant capable of stably providing a position of a wire wound around a fractured femur, the implant including: a body (100) being in a shape extending in a longitudinal direction, and provided with a plurality of locking holes (110) penetrating through the body (100) and being spaced apart from each other at predetermined intervals; a head (200) extending from an upper end of the body (100), and being bent to be in a hook shape for being hooked over a greater trochanter of a femur; a fixed wire support (300) integrally provided in the body (100) or in the head (200), and supporting a wire (700); and a detachable wire support (400) detachably mounted to the body (100), and supporting the wire (700).

### CROSS REFERENCE

This application claims foreign priority under Paris Convention to Korean Patent Application No. 10-2016-0002201, filed 7 Jan. 2016, with the Korean Intellectual Property Office.

### BACKGROUND

The present invention relates to an implant for fixing a femur, the implant capable of stably providing a position of a wire wound around a fractured femur.

Generally, when the greater trochanter or the lesser trochanter of the femur is broken, in order to treat the fractured segment, a wire, a bone plate using screws, or both are used to fix the fractured bone.

However, in the case where the wire is used to fix the fractured bone, it is problematic in that the wire may deviate from a target area in the process of winding the wire around the bone. Further, as time passes, the wire may become unwound or may move.

In the case where the bone plate is used, the screws should be driven into the bone to fix the bone plate to the bone, but in the case where the fractured bone is weak, the bone plate may not be used.

Further, in the case where both the wire and the bone plate are used simultaneously, the wire may not be provided in a desirable area or the wire may become unwound after a procedure.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose an implant for fixing a femur, the implant capable of stably providing a position of a wire wound around a fractured femur.

The present invention is further intended to propose an implant for fixing a femur, the implant allowing a wire to be accurately provided at a desirable location by including a fixed wire support integrally formed in the body and guiding a wire placement location, and a detachable wire support detachably coupled to the body and guiding the wire placement location.

The present invention is furthermore intended to propose an implant for fixing a femur, the implant capable of preventing a wire from being separated therefrom by having contact protrusions provided on each of inner surfaces of through-holes of a fixed or a detachable wire support, into which the wire is inserted, such that the contact protrusions come into close contact with the wire when the wire support is compressed.

In order to achieve the above object, according to one aspect of the present invention, there is provided an implant for fixing a femur, the implant including: a body 100 being in a shape extending in a longitudinal direction, and provided with a plurality of locking holes 110 penetrating through the body 100 and being spaced apart from each other at predetermined intervals; a head 200 extending from an upper end of the body 100, and being bent to be in a hook shape for being hooked over a greater trochanter of a femur; a fixed wire support 300 integrally provided in the body 100 or in the head 200, and supporting a wire 700; and a detachable wire support 400 detachably mounted to the body 100, and supporting the wire 700, wherein the fixed wire support 300 includes either a first wire support 310 being in a hexahedral shape, and disposed between opposite sides of a bent part 210 with a pair of first through-holes 311 penetrating through the opposite sides of the bent part 210, or a second wire support 320 being in a hexahedral shape, and protruding upward between adjacent locking holes 110 of the body 100 with a pair of second through-holes 321 penetrating through opposite sides of the second wire support 320 in directions perpendicular to the longitudinal direction of the body 100, and contact protrusions 500 protrude on each of inner surfaces of the first through-holes 311 or the second through-holes 321 of the fixed wire support 300.

Preferably, the body 100 is configured such that a lower surface thereof is in a curved shape so as to allow opposite edges of the lower surface come into close contact with a surface of a femur.

Preferably, the head 200 includes: a bent part 210 extending from the upper end of the body 100 and being gently bent so as to come into contact with the greater trochanter of the femur; and a hook 220 provided at an end of the bent part 210 to lock the implant by being hooked over the greater trochanter of the femur; and a fixing protrusion 230 provided on a rear surface of the bent part 210, and coming into close contact with a femur, thereby preventing the implant from moving on the femur.

Preferably, the bent part 210 is provided with a locking hole 211 penetrating through an end portion of the bent part 210 to lock the end portion to the femur.

Preferably, the fixed wire support 300 includes a first wire support 310 being in a hexahedral shape, and disposed between opposite sides of a bent part 210 with a pair of first through-holes 311 penetrating through the opposite sides of the bent part 210.

In order to achieve the above object, according to another aspect of the present invention, there is provided an implant for fixing a femur, the implant including: a body 100 being in a shape extending in a longitudinal direction, and provided with a plurality of locking holes 110 penetrating through the body 100 and being spaced apart from each other at predetermined intervals; a head 200 extending from an upper end portion of the body 100, and being bent to be in a hook shape for being hooked over a greater trochanter of a femur; a fixed wire support 300 integrally provided in the body 100 or in the head 200, and supporting a wire 700; and a detachable wire support 400 detachably mounted to the body 100, and supporting the wire 700, wherein the detachable wire support 400 includes: a base part 410 provided on each of opposite sides of each of the locking holes 110 on an upper surface of the body 100; and a locking member 420 being in a hexahedral shape, and detachably coupled to the base part 410, with a pair of third through-holes 421 penetrating through opposite sides of the locking member 420 in directions perpendicular to the longitudinal direction of the body 100.

Preferably, the base part 410 of the detachable wire support 400 includes an oblong groove longitudinally extending along each of the opposite sides of each of the locking holes 110 of the body 100; and the locking member 420 includes a support protrusion 422 inserted into the oblong groove by protruding from a lower surface of the locking member.

Preferably, each of the oblong groove and the support protrusion 422 has a triangular, quadrangular, or semicircular cross-section.

Preferably, the base part 410 of the detachable wire support 400 includes a seat groove 412 being in a cylindrical shape and provided on the opposite sides of each of the locking holes 110; and the locking member 420 includes a protrusion 423 engaged with the seat groove 412 by protruding to be in a cylindrical shape from a lower surface of the locking member.

Preferably, the second wire support 320 or the locking member 420 of the detachable wire support 400 is provided with slits 600 extending from an upper surface thereof to the second through-holes 321 or the third through-holes 421.

Preferably, the wire 700 includes a protection cover 710 covering the wire 700.

Preferably, the protection cover 710 is provided with a wire insert channel 711 in a longitudinal direction of the protection cover.

According to the implant for fixing a femur, it is possible to allow the wire provided by being wound around the femur to be accurately mounted to the target area by both the fixed wire support integrally formed in the body coming into close contact with the femur, and the detachable wire support detachably coupled to the body.

Further, the implant for fixing a femur is capable of preventing the wire from being separated therefrom since the contact protrusions are provided on each of inner surfaces of through-holes of the fixed or the detachable wire support, into which the wire is inserted, such that the contact protrusions come into close contact with the wire when the wire support is compressed.

Further, when the wire support is compressed, it is possible pinch the wire in the through-holes by using small effort by providing the slits extending from an upper surface of the fixed or the detachable wire support to the through-holes, into which the wire is inserted.

### BRIEF DESCRIPTION OF THE DRAWINGS

The above and other objects, features and other advantages of the present invention will be more clearly understood from the following detailed description when taken in conjunction with the accompanying drawings, in which:

FIG. 1A is a perspective front view showing an implant for fixing a femur according to the present invention;

FIG. 1B is a perspective rear view showing the implant for fixing a femur according to the present invention;

FIGS. 2A, 2B, and 2C are sectional views showing a detachable wire support according to a first embodiment of the present invention;

FIG. 3 is a partial perspective view showing a detachable wire support according to a second embodiment of the present invention;

FIG. 4 is an exemplary view showing a fixed wire support according to an embodiment of the present invention;

FIG. 5 is an exemplary view showing a fixed wire support according to another embodiment of the present invention;

FIG. 6 is an exemplary view showing a wire for the implant for fixing a femur according to the present invention; and

FIG. 7 is an exemplary view showing a state where the implant for fixing a femur according to the present invention is used.

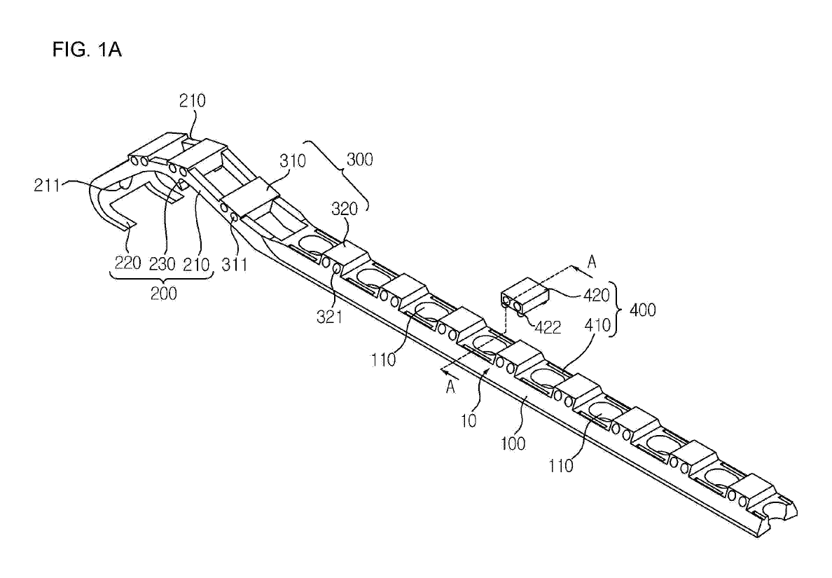

### DETAILED DESCRIPTION OF THE INVENTION

Hereinbelow, exemplary embodiments of the present invention will be described in detail with reference to the accompanying drawings. Throughout the drawings, the same reference numerals will refer to the same or like parts.

As shown in the drawings, an implant 10 for fixing a femur according to the present invention includes: a body 100 coming into contact with a side surface of a femur in a longitudinal direction of the femur; a head 200 extending from an upper end of the body 100, and being bent to be in a hook shape for being hooked over a greater trochanter of the femur; a fixed wire support 300 integrally provided in the body 100 or in the head 200, and supporting a wire 700; and a detachable wire support 400 detachably mounted to the body 100, and supporting the wire 700. The implant further includes a wire 700 fixing the femur by being coupled to the body 100 or the head 200.

The body 100 has a predetermined thickness and is in a shape extending in a longitudinal direction, wherein the body 100 comes into contact with a surface of the femur extending from the greater trochanter in a longitudinal direction. In particular, the body 100 may be made of titanium or titanium alloy to prevent deformation caused by shock during procedure, and preferably, the body 100 has a thickness of 1˜3 cm. Further, it is preferred that the body 100 is configured such that a lower surface thereof is in a curved shape so as to allow opposite edges of the lower surface come into close contact with the surface of the femur.

The body 100 is provided with a plurality of locking holes 110 penetrating through the body 100 and being spaced apart from each other at predetermined intervals. The locking holes 110 are engaged with locking pins that are mounted to the femur to fix the body 100.

The head 200 includes: a bent part 210 extending from the upper end of the body 100 and being gently bent so as to come into contact with the greater trochanter of the femur; and a hook 220 provided at an end of the bent part 210 to lock the implant by being hooked over the greater trochanter of the femur; and a fixing protrusion 230 provided on a rear surface of the bent part 210, and coming into close contact with a femur, thereby preventing the implant from moving on the femur. Here, the bent part 210 extends from opposite edges of the body, and is provided with a locking hole 211 penetrating through an end portion of the bent part 210 to lock the end portion to the femur. By providing the bent part 210, the hook 220, and the fixing protrusion 230 to the body 100, it is possible to ensure contact of the body 100 with the femur and to securely fix the body 100 to the femur. In other words, since the femur is configured such that the greater trochanter defined as the upper portion of the femur is in the form of protrusion, and the rest of the femur longitudinally extends from the greater trochanter, the bent part 210 that is gently bent easily comes into contact with the greater trochanter of the femur, the hook 220 prevents the body 100 from moving down by being hooked over inside the greater trochanter of the femur, and the fixing protrusion 230 prevents the body 100 from moving from side to side by being hooked over the greater trochanter of the femur.

The fixed wire support 300 includes: a first wire support 310 provided in the bent part 210 of the head 200; and a second wire support 320 provided in the body 100.

The first wire support 310 is in a hexahedral shape, and is disposed between opposite sides of a bent part 210 with a pair of first through-holes 311 penetrating through the opposite sides of the bent part 210. The first wire support 310 is configured such that the wire 700 is inserted into a first end of the first through-holes 311, then is wound around the femur, and then is inserted into a second end of the first through-holes 311.

Further, the second wire support 320 is in a hexahedral shape, and protrudes upward between adjacent locking holes 110 of the body 100 with a pair of second through-holes 321 penetrating through opposite sides of the second wire support 320 in directions perpendicular to the longitudinal direction of the body 100. The second wire support 320 is configured such that the wire 700 is inserted into a first end of the second through-holes 321, then is wound around the femur, and then is inserted into a second end of the second through-holes 321. After that, upper portions of the opposite ends of the second through-holes 321 are compressed by a tool such as pliers, whereby the upper portions and the end portions of the second through-holes, namely the inner surfaces of the second through-holes 321, pinch the wire 700.

Meanwhile, the detachable wire support 400 includes: a base part 410 provided on each of opposite sides of each of the locking holes 110 on an upper surface of the body 100; and a locking member 420 being in a hexahedral shape, and detachably coupled to the base part 410, with a pair of third through-holes 421 penetrating through opposite sides of the locking member 420 in directions perpendicular to the longitudinal direction of the body 100. Also, in the case of the locking member 420 of the detachable wire support 400, after the wire 700 is inserted into the third through-holes 421, upper portions of the opposite ends of the third through-holes 421 are compressed by a tool such as pliers, whereby the third through-holes 421 are crushed to pinch the wire 700 between inner surfaces thereof.

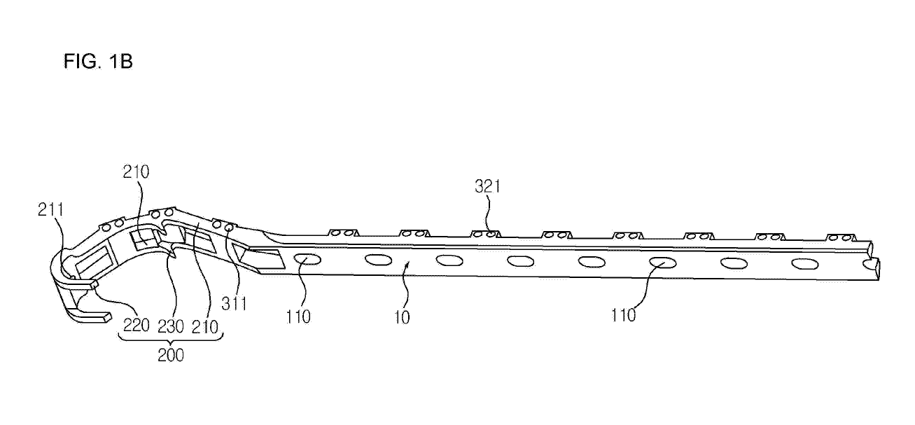

As shown in FIG. 2, according to a first embodiment of the present invention, the base part 410 of the detachable wire support 400 includes an oblong groove longitudinally extending along each of the opposite sides of each of the locking holes 110 of the body 100; and the locking member 420 includes a support protrusion 422 inserted into the oblong groove by protruding from a lower surface of the locking member. Here, it is preferred that each of the oblong groove and the support protrusion 422 has a triangular, quadrangular, or semicircular cross-section.

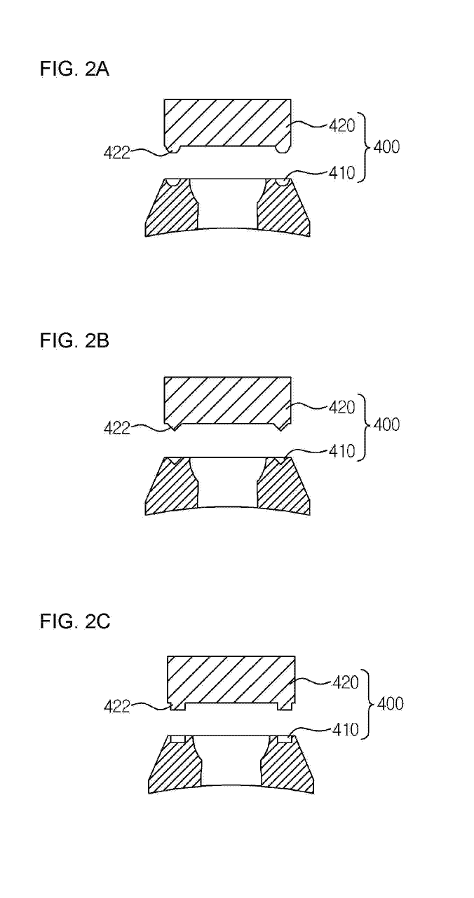

As shown in FIG. 3, according to a second embodiment of the present invention, the base part 410 of the detachable wire support 400 includes a seat groove 412 being in a cylindrical shape and provided on the opposite sides of each of the locking holes 110; and the locking member 420 includes a protrusion 423 engaged with the seat groove 412 by protruding to be in a cylindrical shape from a lower surface of the locking member. Here, it is preferred that the seat groove 412 and the protrusion 423 are each provided four in number.

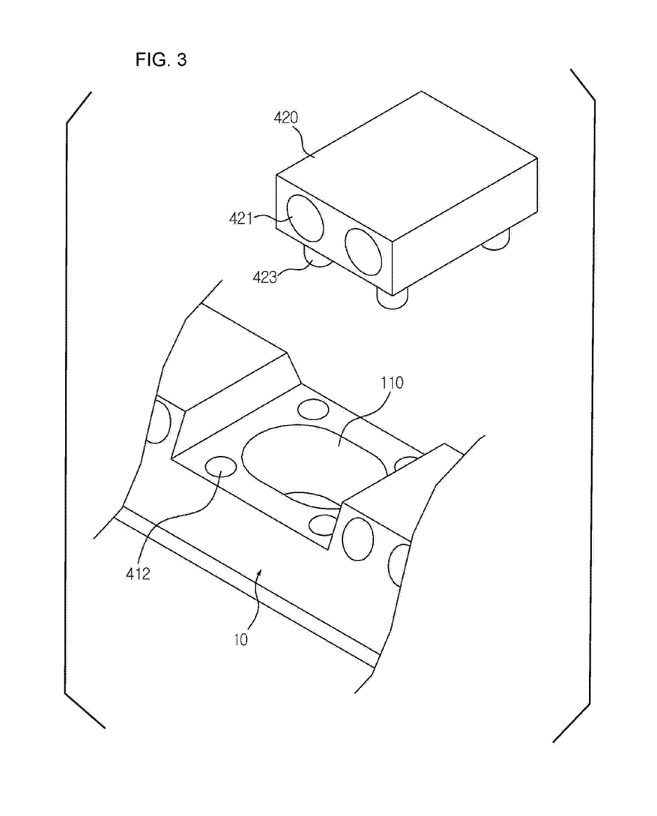

Meanwhile, as shown in FIG. 4, the first and the second through-holes 311 and 321 of the fixed wire support 300 or the third through-holes 421 of the detachable wire support 400 are formed with the contact protrusions 500 on the inner surfaces thereof. Here, it is preferred that the contact protrusions 500 are formed on the left and right inner surfaces of the through-holes, but the contact protrusions may be formed on the entire inner surfaces of the through-holes. Further, it is preferred that the contact protrusions are in a wedge shape inclined in one direction so as to prevent the wire 700 inserted thereinto from moving backwards. The contact protrusions 500 formed on the inner surfaces of the through-holes are locked to the wire 700 when the second and the third through-holes 321 and 421 are compressed by a tool, such as pliers after the wire 700 is inserted into the through-holes.

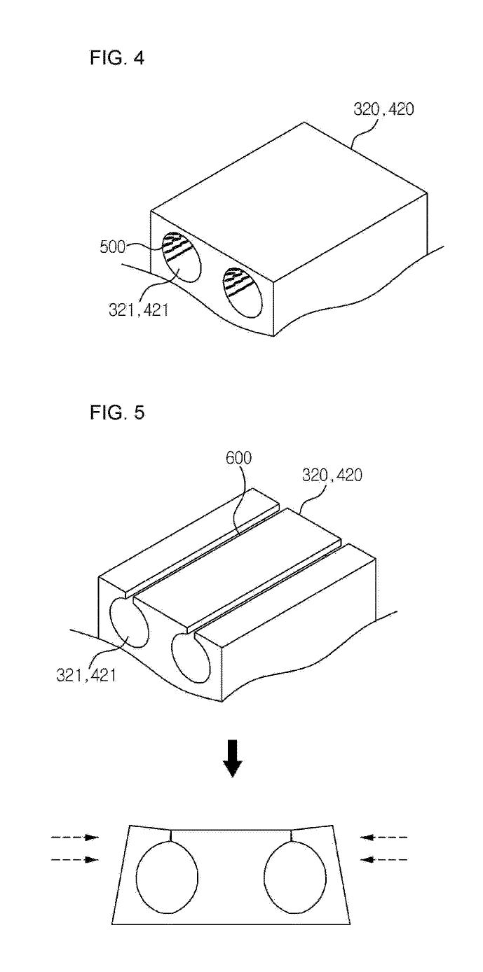

Further, as shown in FIG. 5, the second wire support 320 or the locking member 420 of the detachable wire support 400 is provided with slits 600 extending from an upper surface thereof to the second through-holes 321 or the third through-holes 421. The slits 600 are easily compressed by a tool, such as pliers after the wire 700 is inserted into the second through-holes 321 or the third through-holes 421.

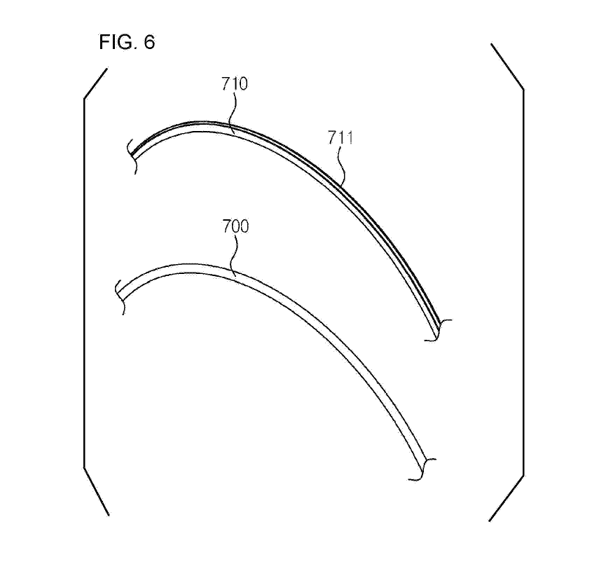

Meanwhile, as shown in FIG. 6, the wire 700 includes a protection cover 710 covering the wire 700 so as to prevent the wire 700 from damaging the femur by coming into contact with the femur. It is preferred that the protection cover 710 is provided with a wire insert channel 711 in a longitudinal direction of the protection cover to be coupled to the wire 700 by a simple manner.

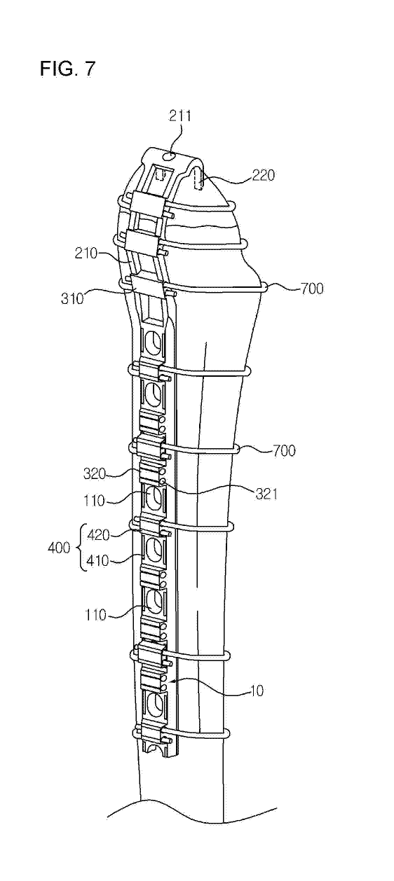

Referring to FIG. 7, the process of fixing the implant for fixing a femur of the present invention is as follows. Firstly, the bent part 210 of the head 200, which is bent at the upper end of the body 100, is placed over the greater trochanter of the femur, and the hook 220 provided at the end of the bent part 210 is hooked over the greater trochanter such that the implant is mounted to the femur. Here, to prevent the body 100 from moving, it is preferred that a locking means, such as a pin, is engaged with the locking hole 211 formed in the bent part 210 or the locking holes 110 formed in the body 100.

Further, in the state where the body 100 comes into close contact with the femur, the wire 700 is coupled to the first wire support 310 of the bent part 210 or the second wire support 320 protruding upward from the body 100, which are defined as locations where the wire is required. In other words, the wire 700 is inserted through a first end of one of the first through-holes 311 or one of the second through-holes 321 of the first and the second wire supports 310 and 320, then is wound around the femur, and then is inserted through a second end of a neighboring through-hole 311, 321, and after that, the opposite ends of the wire 700 are pulled to securely come into close contact with the femur, and are cut thereby finishing the procedure. Here, it is preferred that the wire 700 is provided with the protection cover 710 on a contact surface of the wire, which comes into close contact with the femur, so as to prevent the wire 700 from damaging the femur by coming into contact therewith.

Further, to prevent the wire 700 from slipping out of the first and the second through-holes 311 and 321, upper portions of the second through-holes 321 are compressed by a tool, such as pliers, whereby the second through-holes 321 are crushed to pinch the wire 700 between inner surfaces thereof. Here, it is possible to compress the through-holes to pinch the wire with small effort by providing the slits 600 extending from an upper surface of the second wire support to the second through-holes 321. Further, in the case where the second through-holes 321 are provided with the contact protrusions 500 on the inner surfaces thereof, the contact protrusions 500 are inserted into the surface of the wire 700 when the second through-holes 321 are compressed, thereby preventing the wire 700 from moving backwards.

Meanwhile, in the case where the second wire support 320 is not provided around the locking holes 110 of the body at a location where the wire 700 is required, the locking member 420 of the detachable wire support 400 is mounted to the base part 410 formed in the body 100. After then, the wire 700 is inserted through a first end of one of the third through-holes 421 formed in of the detachable wire support 400, then is wound around the femur, and then is inserted through a second end of a neighboring third through-hole, and after that, the opposite ends of the wire 700 are pulled to securely come into close contact with the femur, and are cut to finish the procedure.

Through the above process, it is possible to accurately place the wire 700 at a desired location.

Thereby, in the case where the bent portion of the femur is required to be treated by using the wire 700, it is possible to accurately place the wire 700 at a desired location.

Although a preferred embodiment of the present invention has been described for illustrative purposes, those skilled in the art will appreciate that various modifications, additions and substitutions are possible, without departing from the scope and spirit of the invention as disclosed in the accompanying claims.

Description of reference characters of important parts

100: body 110: locking holes

200: head 210: bent part

220: hook 230: fixing protrusion

300: fixed wire support 310: first wire support

311: first through-holes 320: second wire support

321: second through-holes 400: detachable wire support

410: base part 412: seat groove

420: locking member 421: third through-holes

422: support protrusion 423: protrusion

500: contact protrusions 600: slits

700: wire 710: protection cover

What is claimed is:

1. An implant for fixing a femur, the implant comprising:
a body (100) being in a shape extending in a longitudinal direction, and provided with a plurality of locking holes (110) penetrating through the body (100) and being spaced apart from each other at predetermined intervals;
a head (200) extending from an upper end of the body (100), and being bent to be in a hook shape for being hooked over a greater trochanter of a femur;
a fixed wire support (300) integrally provided in the body (100) or in the head (200), and supporting a wire (700); and
a detachable wire support (400) detachably mounted to the body (100), and supporting the wire (700), wherein
the fixed wire support (300) includes either a first wire support (310) being in a hexahedral shape, and disposed between opposite sides of a bent part (210) with a pair of first through-holes (311) penetrating through the opposite sides of the bent part (210), or a second wire support (320) being in a hexahedral shape, and protruding upward between adjacent locking holes (110) of the body (100) with a pair of second through-holes (321) penetrating through opposite sides of the second wire support (320) in directions perpendicular to the longitudinal direction of the body (100), and
contact protrusions (500) protrude on each of inner surfaces of the first through-holes (311) or the second through-holes (321) of the fixed wire support (300).

2. The implant for fixing a femur of claim 1, wherein
the body (100) is configured such that a lower surface thereof is in a curved shape so as to allow opposite edges of the lower surface come into close contact with a surface of a femur.

3. The implant for fixing a femur of claim 1, wherein the head (200) includes:
a bent part (210) extending from the upper end of the body (100) and being gently bent so as to come into contact with the greater trochanter of the femur; and
a hook (220) provided at an end of the bent part (210) to lock the implant by being hooked over the greater trochanter of the femur; and
a fixing protrusion (230) provided on a rear surface of the bent part (210), and coming into close contact with a femur, thereby preventing the implant from moving on the femur.

4. The implant for fixing a femur of claim 3, wherein
the bent part (210) is provided with a locking hole (211) penetrating through an end portion of the bent part (210) to lock the end portion to the femur.

5. The implant for fixing a femur of claim 1, wherein
the second wire support (320) is provided with slits (600) extending from an upper surface of the second wire support to the second through-holes (321).

6. An implant for fixing a femur, the implant comprising:
a body (100) being in a shape extending in a longitudinal direction, and provided with a plurality of locking holes (110) penetrating through the body (100) and being spaced apart from each other at predetermined intervals;
a head (200) extending from an upper end portion of the body (100), and being bent to be in a hook shape for being hooked over a greater trochanter of a femur;
a fixed wire support (300) integrally provided in the body (100) or in the head (200), and supporting a wire (700); and
a detachable wire support (400) detachably mounted to the body (100), and supporting the wire (700), wherein
the detachable wire support (400) includes: a base part (410) provided on each of opposite sides of each of the locking holes (110) on an upper surface of the body (100); and a locking member (420) being in a hexahedral shape, and detachably coupled to the base part (410), with a pair of third through-holes (421) penetrating through opposite sides of the locking member (420) in directions perpendicular to the longitudinal direction of the body (100).

7. The implant for fixing a femur of claim 6, wherein
the base part (410) of the detachable wire support (400) includes an oblong groove longitudinally extending along each of the opposite sides of each of the locking holes (110) of the body (100); and the locking member (420) includes a support protrusion (422) inserted into the oblong groove by protruding from a lower surface of the locking member.

8. The implant for fixing a femur of claim 7, wherein
each of the oblong groove and the support protrusion (422) has a triangular, quadrangular, or semicircular cross-section.

9. The implant for fixing a femur of claim 6, wherein
the base part (410) of the detachable wire support (400) includes a seat groove (412) being in a cylindrical shape and provided on the opposite sides of each of the locking holes (110); and the locking member (420) includes a protrusion (423) engaged with the seat groove (412) by protruding to be in a cylindrical shape from a lower surface of the locking member.

10. The implant for fixing a femur of claim 6, wherein
contact protrusions (500) protrude on each of inner surfaces of the third through-holes (421) of the detachable wire support (400).

11. The implant for fixing a femur of claim 1, wherein
the locking member (420) of the detachable wire support (400) is provided with slits (600) extending from an upper surface of the locking member to the third through-holes (421).

12. An implant for fixing a femur, the implant comprising:
a body (100) being in a shape extending in a longitudinal direction, and provided with a plurality of locking holes (110) penetrating through the body (100) and being spaced apart from each other at predetermined intervals;
a head (200) extending from an upper end portion of the body (100), and being bent to be in a hook shape for being hooked over a greater trochanter of a femur;
a fixed wire support (300) integrally provided in the body (100) or in the head (200), and supporting a wire (700); and
a detachable wire support (400) detachably mounted to the body (100), and supporting the wire (700), wherein
the wire (700) includes a protection cover (710) covering the wire (700), and
the protection cover (710) is provided with a wire insert channel (711) in a longitudinal direction of the protection cover.
